(19) United States
(12) Patent Application Publication
(10) Pub. No.: US 2025/0090763 A1
(43) Pub. Date: Mar. 20, 2025

(54) PRECISION SYRINGE ACTUATOR SYSTEM

(71) Applicant: ClearPoint Neuro, Inc., Solana Beach, CA (US)

(72) Inventors: Robert E. Kearney, San Diego, CA (US); Nathan Williams, San Diego, CA (US); Nolan Zaayer, Paso Robles, CA (US)

(21) Appl. No.: 18/886,211

(22) Filed: Sep. 16, 2024

Related U.S. Application Data
(60) Provisional application No. 63/582,951, filed on Sep. 15, 2023.

(51) Int. Cl. A61M 5/315

(52) U.S. Cl. CPC A61M 5/31576

(57) ABSTRACT

A syringe adapter driver system includes a housing configured to receive a syringe device and an actuator attached to the housing, the actuator configured to drive a plunger assembly to cause the plunger assembly to extend or retract through a barrel of the syringe and expel or receive therapeutic. A syringe driver assembly removably attaches the plunger assembly to the housing, wherein the syringe driver assembly moves relative to the housing. A lead screw mechanically couples the actuator to the syringe driver assembly.

### CROSS REFERENCE TO RELATED APPLICATION

The present application claims priority to U.S. Provisional Application No. 63/582,951, filed on Sep. 15, 2023, and entitled “PRECISION SYRINGE DRIVER,” the entirety of which is incorporated by reference herein.

### BACKGROUND

In situations where a therapeutic is delivered to a patient, many therapeutics have a specified volume that must be delivered over a set period. Current systems utilize an infusion pump for delivery or utilize delivery by hand using a syringe.

Gene therapies are commonly delivered using an infusion pump (such as a syringe pump.) There are generally two scenarios in relation to an MR type solution: 1) deliver the therapeutic by placing an MR Conditional infusion pump in an MRI suite a sufficient distance away from MR magnet; or (2) insert a therapeutic delivery device to target under MRI guidance, then relocate the patient outside the MRI suite to deliver the therapeutic with a syringe pump. The first scenario often results in a large dead volume as the cannula must be sufficiently long to reach from the infusion pump to the iso center of the magnet. The second scenario often results in possible error in therapeutic delivery if the placement of the cannula or delivery device is changed between initial placement and the start of the infusion. Both scenarios have challenges given that therapeutics can cost millions of dollars per dose. Less dead volume for the therapeutic leads to lower cost. In addition, the accuracy of the placement of the cannula tip may be impacted as the patient is moved such that the second scenario requires precise accuracy, which can also present risks. However, it should be appreciated that the features described herein are not limited to an MRI specific solution but can be useful wherever a small volume, precise volume or slow flowrate are desirable and using an automated pump is impractical or undesirable.

For cell therapies, which are commonly delivered by hand, the delivery syringe unitizes graduations or other markings for determining volume. Or a custom device is used to deposit the desired volumes. Such manual infusion requires precise control of the syringe plunger position to deliver small volumes and may include a time interval for the cell delivery. With existing systems, it can be challenging to repeatedly deliver the same volume of therapeutic as the volume delivered is dependent on the plunger position. In this case, a small movement of the plunger can result in a relatively large impact to volume delivered.

### SUMMARY

There is a need for a therapeutic delivery system, such as a syringe delivery system, that can reliably deliver precise volumes of therapeutic to a patient. Disclosed herein is a syringe delivery system having a plunger (or other actuator) that can be reliably and repeatedly be advanced or retracted a precision set distance using position indicators of the system. For gene therapies with low volumes where convection enhanced delivery is not critical, a dead volume can be reduced using the systems described herein. Additionally, a dead volume of a cannula of the system can be reduced significantly as the system can be configured such that it is not made of magnetic components and can be located close to a magnet of an MR system.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The details of one or more variations of the subject matter described herein are set forth in the accompanying drawings and the description below. Other features and advantages of the subject matter described herein will be apparent from the description and drawings, and from the claims.

FIG. 1 shows a perspective view of a therapeutic delivery system that includes a syringe.

FIG. 2 shows a cross-sectional view of a rack and pinion assembly of the therapeutic delivery system.

FIG. 3 shows a perspective view of a therapeutic delivery system that includes a syringe having multiple gears in a rack and pinion system.

FIG. 3A shows a perspective view of a therapeutic delivery system having an alternate actuation assembly.

FIG. 4 shows a front view of a actuation knob of the system.

FIG. 5 shows a side view of the actuation knob mounted on a plate.

FIG. 6 shows an alternate embodiment of the syringe system that uses a rotatable collar to advance/retract a plunger assembly.

FIG. 7 shows an alternate embodiment of the syringe system.

FIG. 8 shows an alternate embodiment wherein the system includes an adaptor assembly that is configured to be docked to an existing syringe device.

FIGS. 9 and 10 shows examples of actuation knobs.

FIG. 11 shows another embodiment of the adapter assembly.

FIGS. 12 and 13 show additional embodiments of the adapter assembly.

FIG. 14 shows an additional embodiment of the adapter assembly.

FIGS. 15 and 16 show an alternate embodiment wherein the system includes an adaptor assembly that is configured to be docked to an existing syringe device.

FIG. 17 shows an enlarged view of a portion of the adaptor assembly of FIGS. 15 and 16.

FIG. 18 shows an alternate embodiment of an adaptor assembly.

FIG. 19 shows an enlarged view of a portion of the adaptor assembly of FIG. 18.

FIGS. 20-22 show another embodiment.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

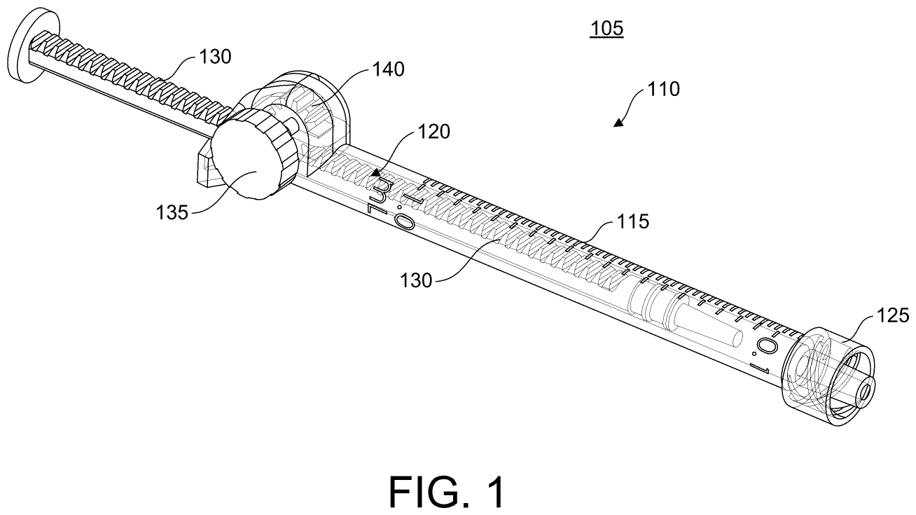

FIG. 1 shows a perspective view of a therapeutic delivery system 105 that includes a syringe 110 formed of a barrel 115 in which a plunger assembly 120 is slidably positioned. The barrel 115 has a distal coupler 125 that can be removably attached to a therapeutic delivery device, such as a cannula, needle, etc, wherein the delivery device can be coupled to a patient for delivering therapeutic from the syringe 110 to the patient. Delivery of therapeutic via the delivery device can accomplished via various mechanisms including, for example, movement of a seal/piston (as described below) or moving a wire/mandrel that extends into the delivery device (such as for front-loading of therapeutic into the barrel). The barrel defines an internal volume that can contain an initial amount of therapeutic.

The plunger assembly 120 includes an elongated body 130 such as a rod slidably positioned inside the barrel 115. A seal or piston (which is in a sealed relationship with an inner wall of the barrel) can be positioned on a distal end of the elongated body 130 for pushing therapeutic out of the barrel via the distal coupler and into the attached therapeutic delivery device (or for pulling therapeutic into the barrel via the distal coupler and from the attached therapeutic delivery device.) An actuator, such as a roller wheel 135, is attached to the elongated body 130 and is configured to be actuated to slide the elongated body further into or out of the barrel 115. In an non-limiting example, the actuation knob is a roller wheel although the actuation knob can be any of a variety of actuation structures. The actuation knob 135 can be mechanically coupled to the elongated body 130 in a variety of manners such as via a gear system (combined with a rack and pinion system) such that rotation of the actuation knob 135 causes a predetermined and corresponding linear movement of the elongated body 130 in a calibrated manner.

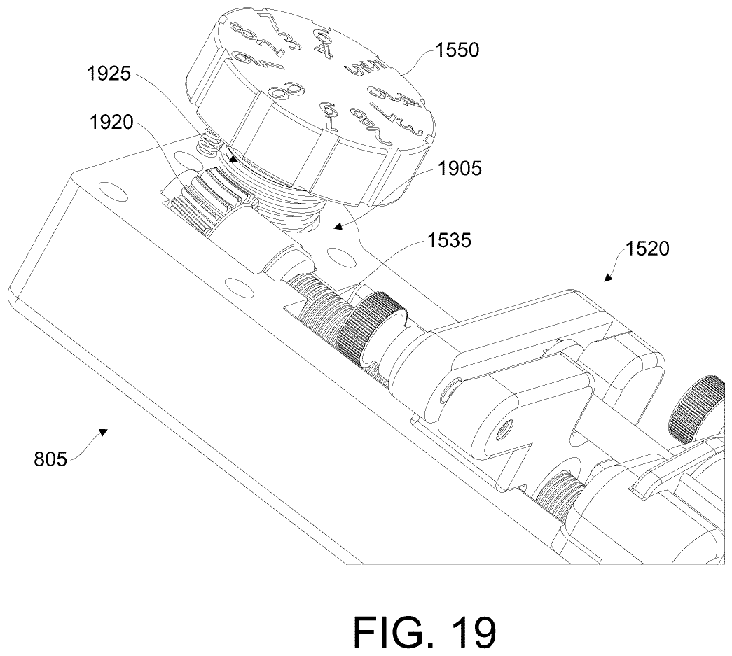

Each rotation of the actuation knob 130 can be correlated to a specific volume transfer of therapeutic volume into or out of the barrel 115. In a non-limiting example, 1 full turn of the actuation knob 130 results ejection of a predetermined amount of therapeutic from the barrel such as delivery of 10 μL therapeutic to a target location via the attached therapeutic delivery device. The barrel 115 can have an inner diameter (ID), and gear ratio(s) of the device can be predetermined to achieve the requirements of a particular application. The gear rations can vary. In non-limiting examples, the ratio is 2:1, 1:1, or 1:2 for the rack and pinion. In other non-limiting examples, the ratio is 10:1 or 20:1 for a worm gear configuration (such as shown in FIG. 19.

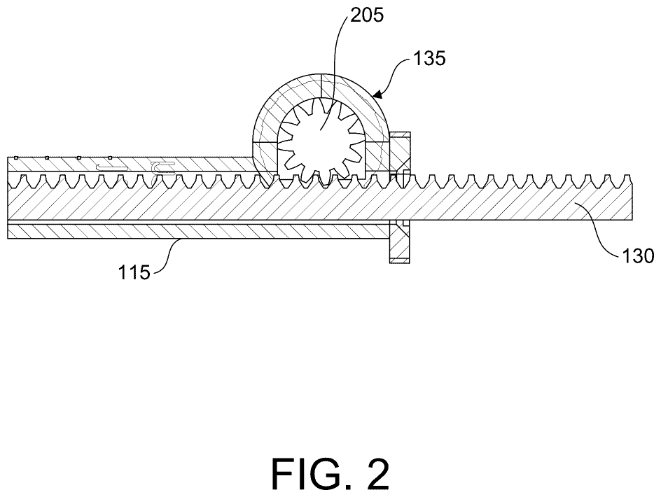

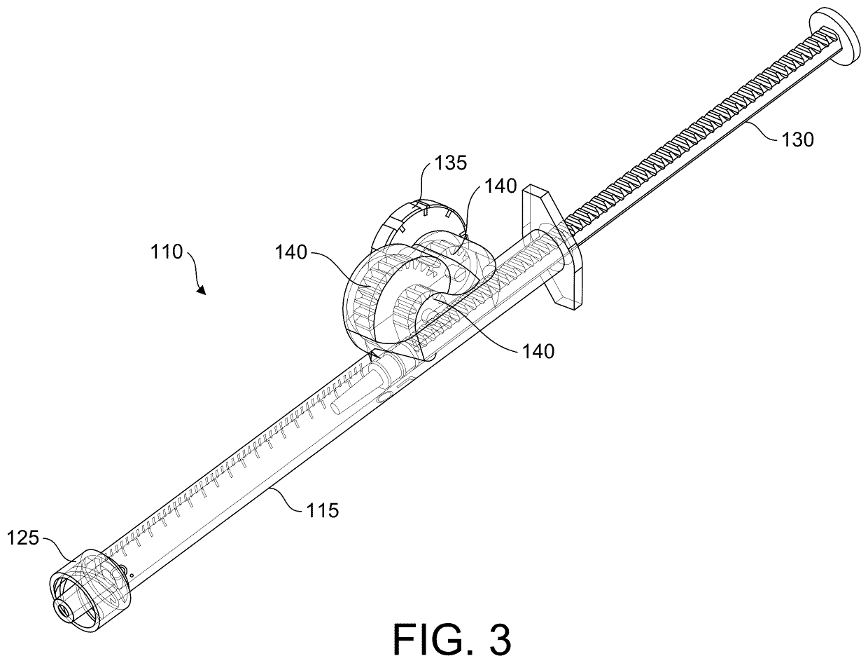

As mentioned, the actuation knob 135 is mechanically coupled to the elongated body 130 such that rotation of the actuation knob 135 causes calibrated linear translation of the elongated body 130. As shown in FIG. 2, this can be accomplished vis a rack and pinion system wherein the elongated body 130 is a rack mechanically coupled to a single gear 140 comprising a pinion 205 coupled to the actuation knob 135 to achieve mechanical linkage therebetween. The rack and pinion system can vary to achieve any of a wide variety of correspondence between rotation of the actuation knob 135 and translation of the elongated body 130 (and resulting volumetric transfer of therapeutic.) In the embodiment of FIG. 1, the rack and pinion system includes a single gear 140 coupled to the actuation knob 135. FIG. 3 shows an example where the system includes several gears 140 configured to achieve a predetermined transfer of therapeutic based on a full or partial rotation of the actuation knob 135.

With reference to FIG. 3A, the system can be configured such it utilizes a ‘worm type’ gear assembly 350 (i.e., a single gear) instead of a pinion to drive the elongated body 130 of the plunger assembly. The gear assembly 350 is configured such that it can be removed/disengaged so that the plunger the elongated body 130 of the plunger assembly can move freely relative to the barrel 130. That is, the gear assembly 350 is removably attached to the barrel 130. In this manner, a first gear assembly 350 can be replaced with a different, second gear assembly.

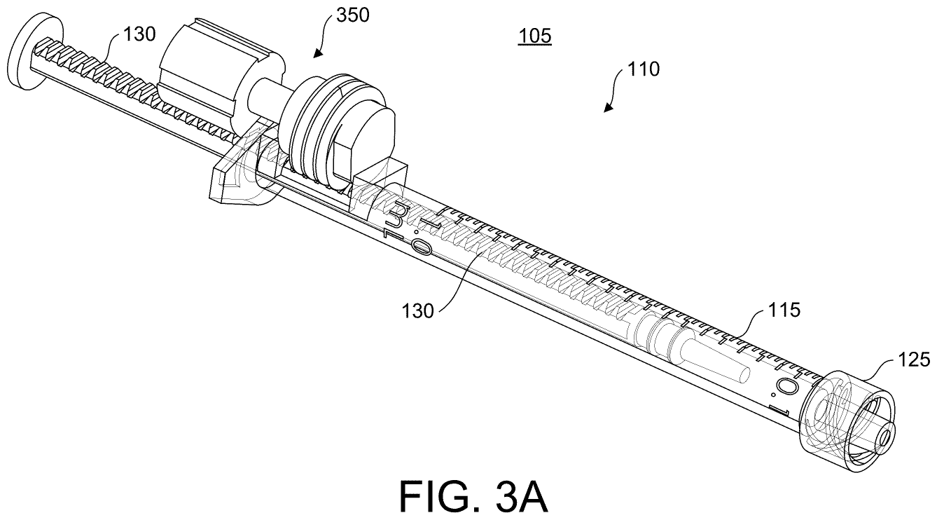

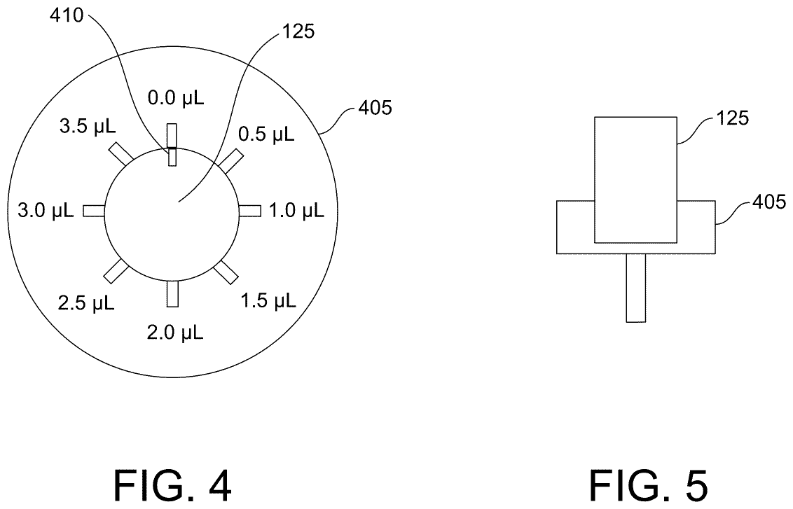

The rack and pinion and gears can be configured such that a full or partial turn of the actuation knob 125 achieves calibrated delivery of a predetermined volume of therapeutic. FIGS. 4 and 5 show front views of the actuation knob 125, which is mounted on a plate 405 or other structure wherein the actuation knob and/or plate include calibrated markings that provide an indication to a user of volume delivered based on a full or partial turn of the actuation knob 125. The actuation knob 125 includes an indicator 410 (such a marker) that can be rotatably aligned with the calibrated marking on the plate 405 to indicate the volume delivered for a full or partial turn.

The actuation knob and/or the rack and pinion system can include one or more mechanisms configured to provide tactile feedback for when the plunger assembly has been advanced a predetermined amount and in turn delivered a specific volume. This can be accomplished in a variety of ways including, for example, a raised section or indented portion on the actuation knob 125 or stationary housing of the system. The actuation knob or gears can be connected to a motor wherein the motor automatically retracts or advance the plunger assembly to withdraw or infuse therapeutic substances respectively. The motor can be configured to control the flow rate and volume delivered for accurate dosing. The system can be MR Conditional or MR Safe.

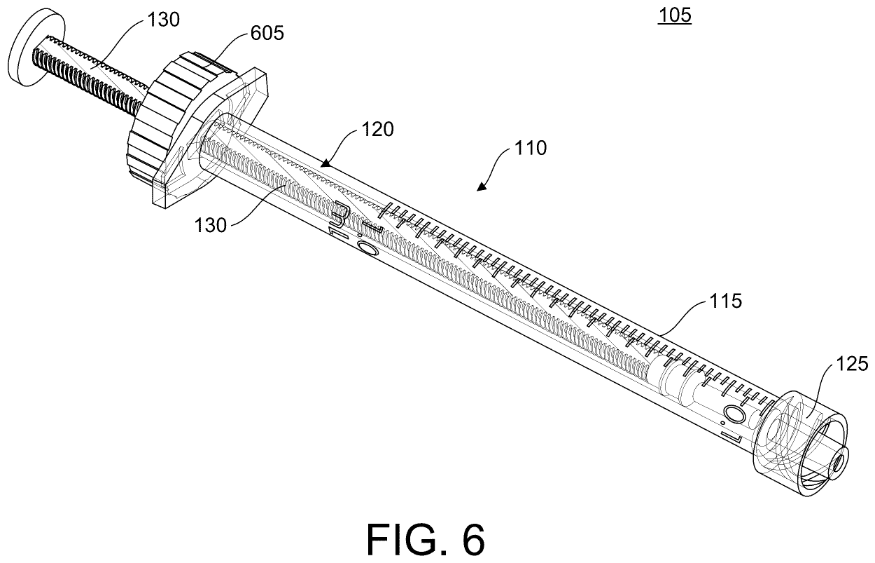

FIG. 6 shows an alternate embodiment of the syringe system that uses a rotatable collar 605 to advance/retract the elongated body 130 of the plunger assembly 120. The collar 605 rotates about an axis that is parallel to a long axis of the barrel 115. This embodiment can provide additional precision but may require additional rotations of the collar 605 to deliver the required volume. The collar 605 is mechanically coupled to the elongated body 130 via a lead screw type mechanism rather than a rack and pinion system of the prior embodiment. The collar and/or a gear may be configured such that it can be removed or disengaged from the elongated body 130 so that the elongated body of the plunger assembly can move freely relative to the barrel 130.

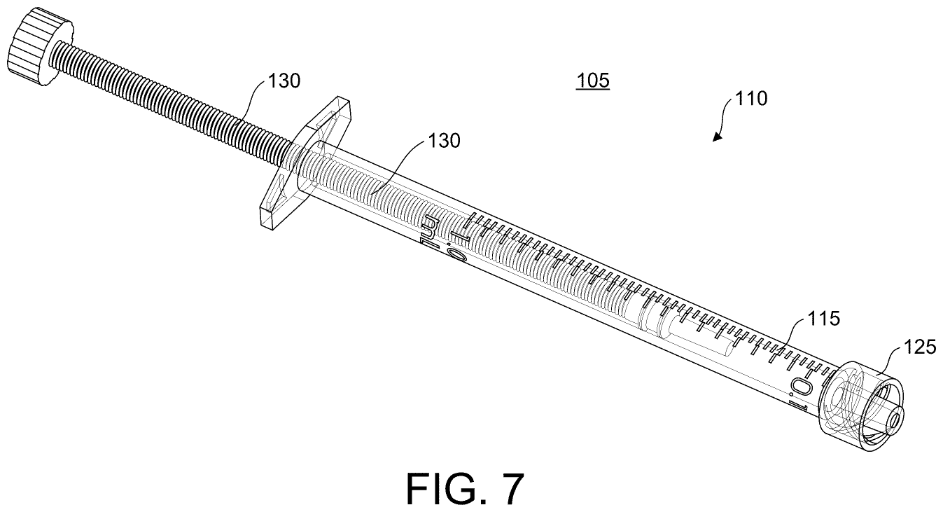

FIG. 7 shows an alternate embodiment of the syringe system. The elongated body 130 the plunger assembly has external threads that mechanically engage with complementary threads on an inner surface of the barrel 115. Or the complementary threads can be on a separate component that is coupled to the barrel 115. The elongated body 130 is thus rotated to achieve advancement or retraction relative to the barrel 115. The threading may be configured such that it can be removed/disengaged so that the elongated body 130 can move freely relative to the barrel 115.

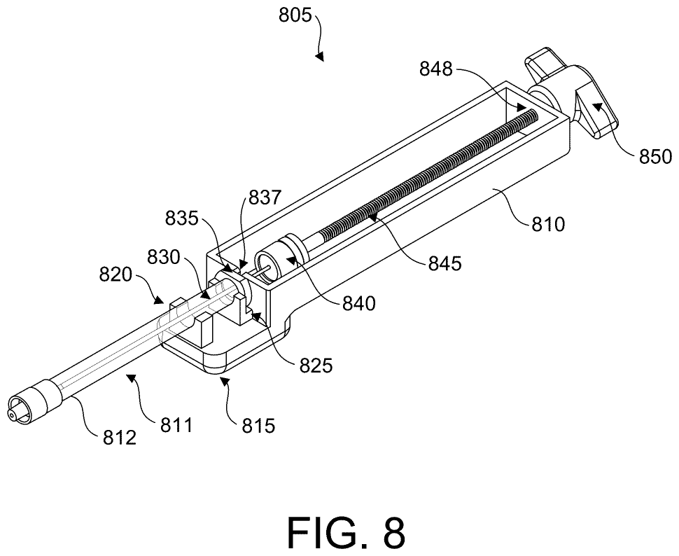

FIG. 8 shows an alternate embodiment wherein the system includes an adaptor assembly 805 that is configured to be docked to an existing syringe device 811 having a barrel 812. The adaptor assembly 805 mechanically separates the advancement mechanism from the syringe device 811. It can use a leadscrew or other gear mechanism to move a stage that forcibly interacts with the syringe device to advance or retract a plunger assembly of the syringe device 811. An example advantage of this embodiment is the syringe device 811 can be loaded as a normal syringe, then the therapeutic can be delivered with high accuracy through the lead screw pitch of the adaptor assembly 805. It also allows for an off-the-shelf syringe (e.g. BD 3 ml syringe) to be used instead of requiring a custom syringe. The adaptor assembly 805 has a handle or knob that provides another way to indicate the volume delivered with each turn.

With reference still to FIG. 8, the adapter assembly 805 includes a housing 810 that has a dock region 815 including a mounting clip 820 and a flange wedge or clip 825 to which the syringe device 811 can be docked. The syringe device 811 has a plunger or elongated body 830. A finger flange 835 of the syringe device 811 forms an opening 837 through which the elongated body 830 extends into the adapter assembly 805. The adapter assembly 805 includes a plunger cap 840 that attaches to the elongated body 830 and that is attached to a lead screw 845 that can be extended or retracted using a handle or knob 850 that rotates the lead screw 845. The lead screw 845 engages a threaded hole 848 of the adapter assembly 805 such that the lead screw 845 advances or retracts based on the rotational direction of the knob 850.

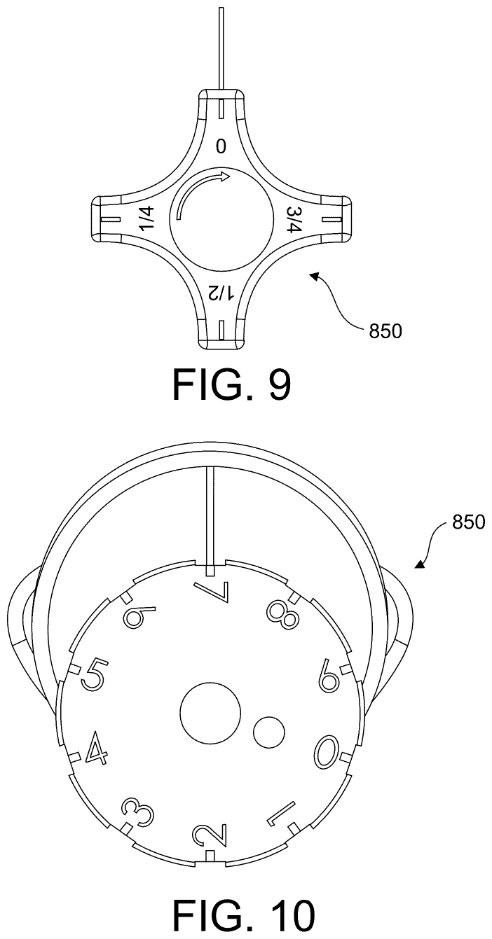

In use, the knob 850 is rotated to cause rotation of the lead screw 845, which results in corresponding extension or retraction of the attached elongated body of the syringe device 811. This in turn causes the syringe device 811 to expel or receive therapeutic. The knob 850 can be configured so visual and tactile indications allow the user to determine the volume delivered or received therapeutic. FIG. 9 shows an example of the actuation knob 850 having one or more volumetric delivery indicators. FIG. 10 shows another example of an actuation knob 850.

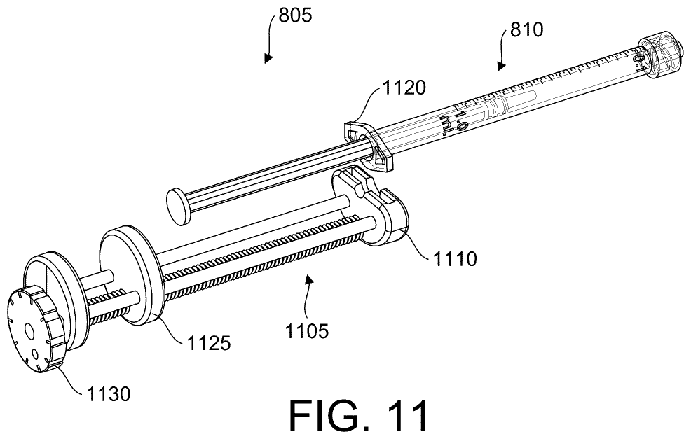

FIG. 11 shows another embodiment of the adapter assembly 805 wherein the adapter assembly 805 is formed of a frame 1105 having a dock 1110 that receives a flange 1120 of the syringe device 811. The adapter assembly 805 has a coupler such as a stage 1125 that causes extension or retraction of the plunger assembly of the syringe device 811 based on actuation of a knob 1130.

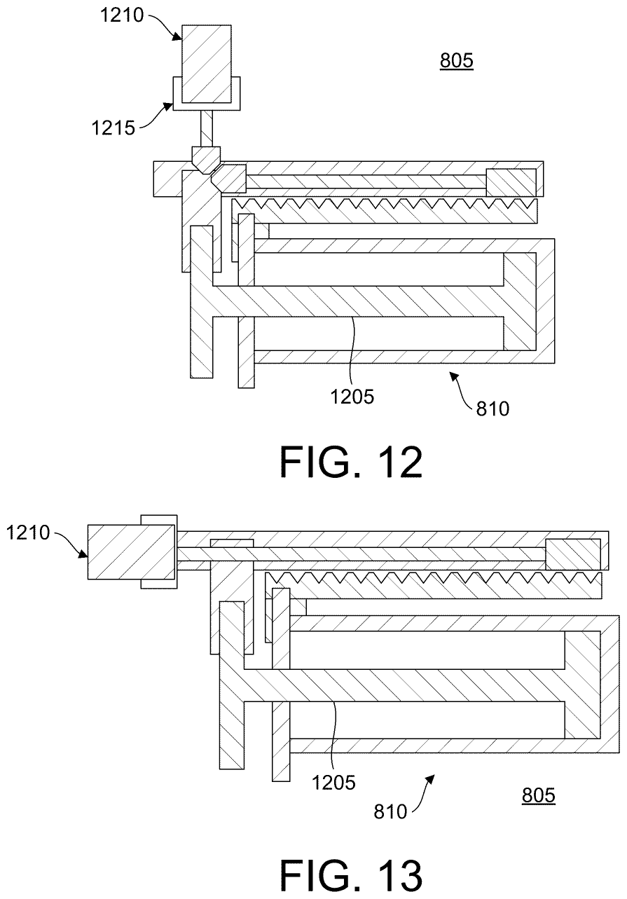

FIGS. 12 and 13 show additional embodiments of the adapter assembly 805 attached to a schematic representation of the syringe device 811 having a plunger assembly 1205. This embodiment uses a system of one or more gears to advance or retract the plunger assembly 1205. This system maintains a controlled delivery via indications on a thumbwheel 1210 of the adapter assembly 805. For example, the thumbwheel may be attached to an indicator structure 1215 such as the plate 405 described above with reference to FIGS. 4 and 5. The thumbwheel 1210 can be mounted perpendicularly relative to the syringe device 811 (as shown in FIG. 12), or in parallel with the syringe device 811 (as shown in FIG. 13) depending on the application. The embodiments of FIGS. 12 and 13 can be more compact or smaller than the other embodiments of the adapter device.

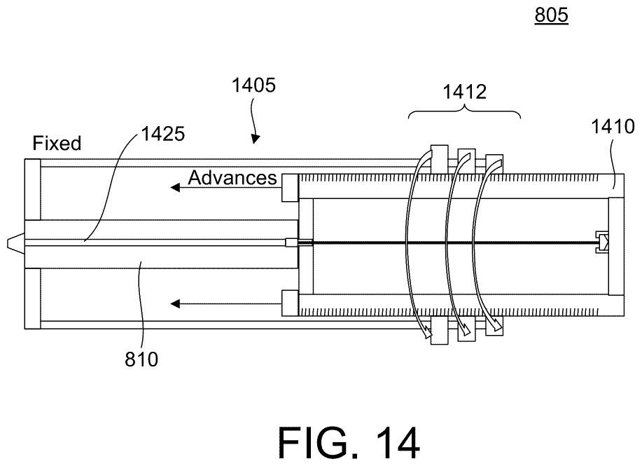

In another embodiment of the adapter assembly 805 shown in FIG. 14, the adapter assembly 805 includes an external housing 1405 with a plurality of knobs 1412. Each knob corresponds to a specific ratio of plunger displacement upon actuation of the respective knob. A plunger housing 1410 has external threads that mechanically interact with threads of the knobs such that each turn of the knobs drives a plunger 1425 forwards or backwards. The knobs can achieve various displacement (e.g. one turn of the small knob is 10 μL per rotation and one turn of the large knob is 100 μL per rotation). This can be accomplished via gear ratios or by utilizing different, discrete mechanical features to accomplish the movement for each knob.

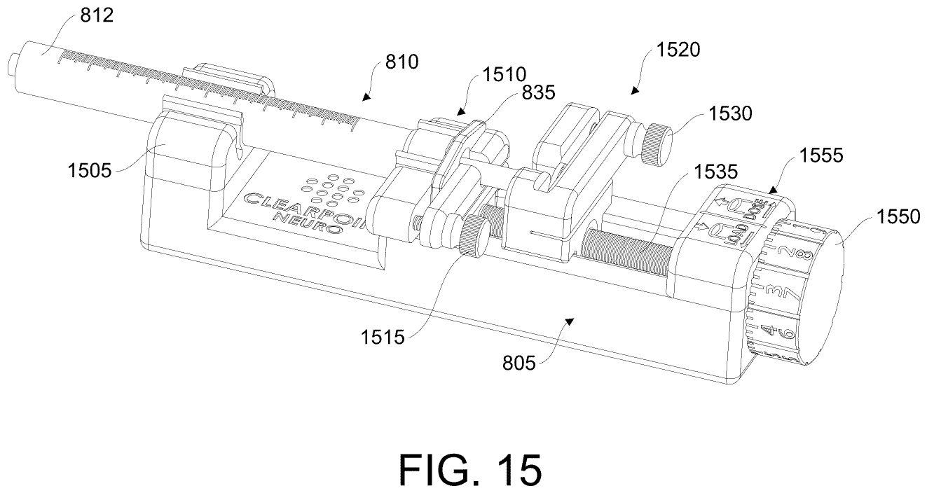

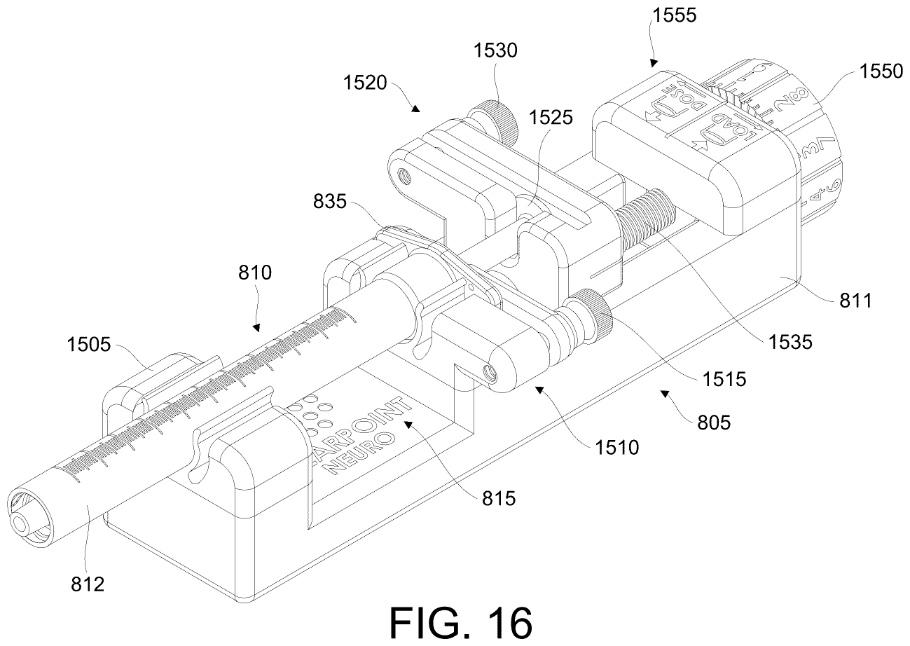

FIGS. 15 and 16 show an alternate embodiment wherein the system includes an adaptor assembly 805 that is configured to be docked to an existing syringe device 811 having a barrel 812. The adapter assembly 805 includes a housing 810 that has a dock region 815 with an upwardly extending structure that forms a seat 1505 sized and shaped to receive the barrel 812 and secure the barrel 812 in a fixed position on the housing. A syringe holder assembly 1510 removably attaches to a portion of the syringe device 811 such as to a finger flange 835 of the syringe device 811. The syringe holder assembly 1510 can include a first portion and a second portion that hold the finger flange 835 therebetween and that can be tightened to the finger flange 835 via one or more thumb screws 1515. The seat 1505 and the syringe holder assembly 1510 hold the syringe barrel 812 in a fixed position relative to the housing 810 of the adaptor assembly 805.

A syringe driver assembly 1520 attaches to the syringe's plunger assembly such as to a plunger flange 1525 (FIG. 16) on a proximal end of the plunger assembly. As mentioned, the plunger assembly includes an elongated body slidably positioned inside the syringe barrel with a seal or piston inside the barrel for pushing therapeutic out of the barrel via the distal coupler and into the attached therapeutic delivery device (or for pulling therapeutic into the barrel via the distal coupler and from the attached therapeutic delivery device.). The syringe driver assembly 1520 can be tightened to the plunger flange 1525 via one or more thumb screws 1530.

The syringe driver assembly 1520 is threadedly attached to a drive screw or lead screw 1535 that can rotate using an actuator such as a handle or knob 1550. The lead screw 845 engages a threaded hole of the plunger assembly 120 such that plunger assembly 120 advances or retracts based on a rotational direction of lead screw 845 via the knob 1550. The adaptor assembly 805 can include or one more markers or indicators that provide a user with an indication as to how actuation of the knob 850 affects the syringe device. For example, the adaptor assembly 805 can include indicators 1555 that indicate to a user that clockwise rotation of the knob 1550 results in the syringe device dosing or ejecting fluid while counterclockwise rotation of the knob 1550 results in the syringe device loading or receiving fluid. Furthermore, the knob 1550 can include one or more markers or volumetric delivery indicators along its circumference that provide an indication as to how much fluid is expelled or received by the syringe device based upon rotation of the knob 1550. In a non-limiting example, a full rotation of the knob 1550 corresponds to 10 μL of fluid transfer.

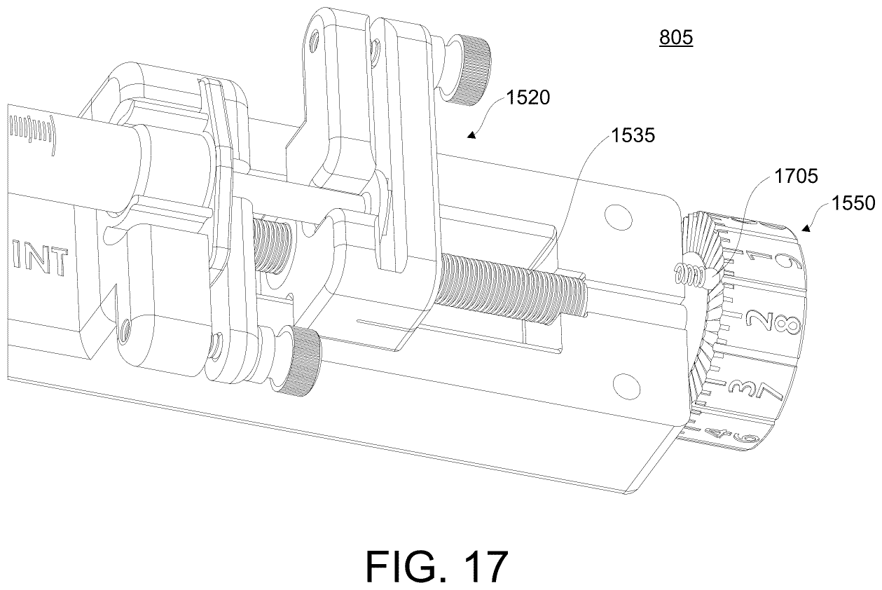

FIG. 17 shows an enlarged view of the adaptor assembly 805 with a cover portion removed to show the lead screw 1535, which is directly attached to the knob 1550. Thus, rotation of the knob 1550 directly drives the lead screw 845 and the syringe driver assembly 1520. It should be appreciated that a gearing assembly can be coupled to the knob 1550 and the lead screw 845. The knob 1550 can be configured so visual and tactile indications allow the user to determine the volume delivered or received therapeutic. For example, the knob 1550 can be mechanically coupled to a spring and ball 1705 that are biased toward teeth or ratchet structures on an inner surface of the knob 1550. As the knob rotates, the ball 1705 alternately springs in and out of the teeth to emit a feedback sound (such as a clicking sound) that indicates to a user that the knob 1550 is being incrementally rotated. In a non-limiting example, each feedback sound (such as each click) corresponds to 0.2 μL of fluid transfer for the embodiment of FIG. 17 and 0.05 μL of fluid transfer for the embodiment of FIG. 19.

In use, the knob 1550 is rotated to cause rotation of the lead screw 1535, which results in corresponding extension or retraction of the syringe driver assembly 1520 as well as the plunger of the syringe device. This in turn causes the syringe device 811 to expel or receive therapeutic.

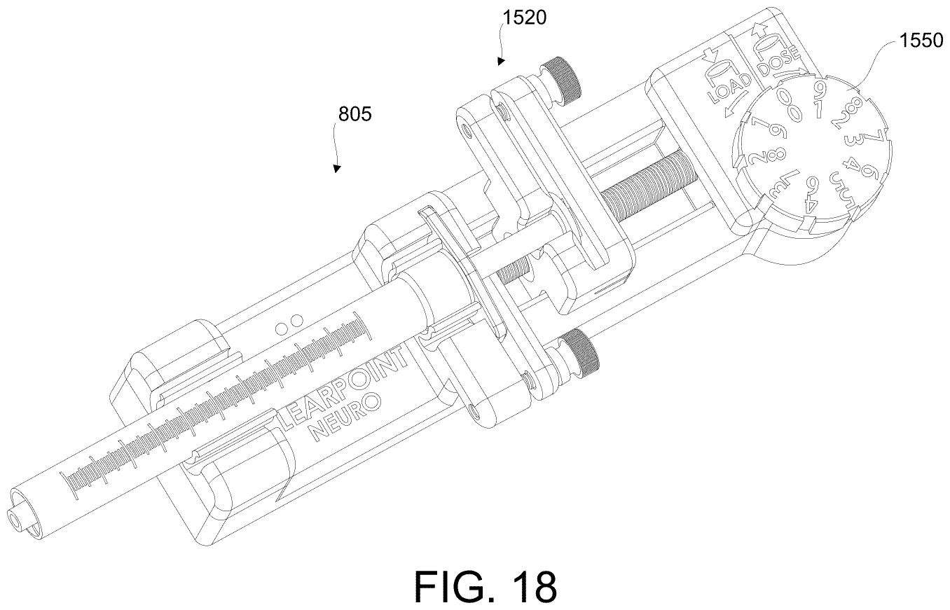

FIG. 18 shows another embodiment of the adaptor assembly 805 wherein the knob 1550 is upwardly facing. This embodiment includes a worm gear assembly to drive the syringe driver assembly 1520 via rotation of the knob 1550. FIG. 19 shows an enlarged view of the adaptor assembly 805 with a cover portion removed to show the lead screw 1535, which mechanically coupled to the knob 1550 via a worm gear assembly 1905. The lead screw 1535 has a toothed portion 1920 that mechanically engages a spiral thread 1925 of the knob 1550 to achieve rotation of the lead screw 1535 via knob rotation. In a non-limiting example, a full rotation of the knob 1550 corresponds to 1 μL of fluid transfer.

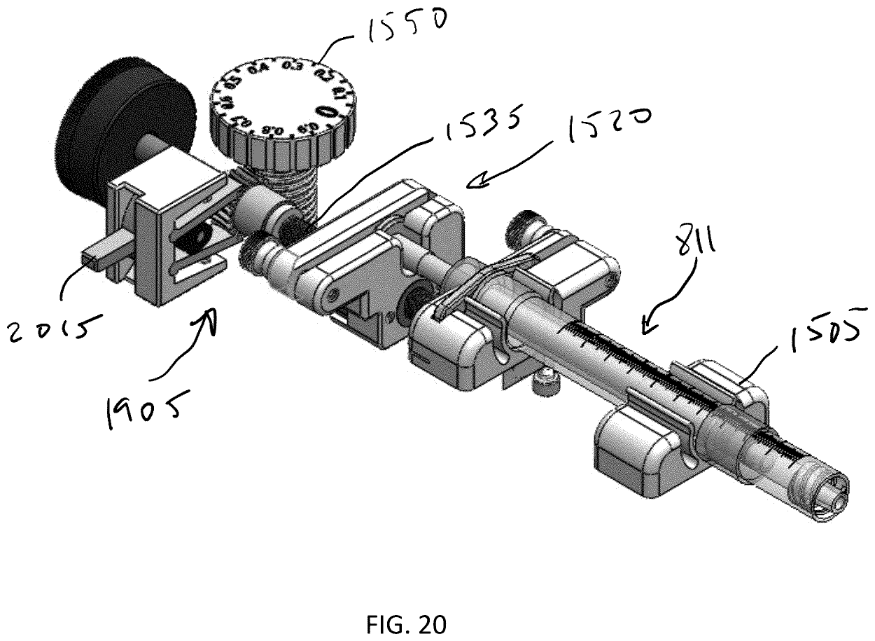

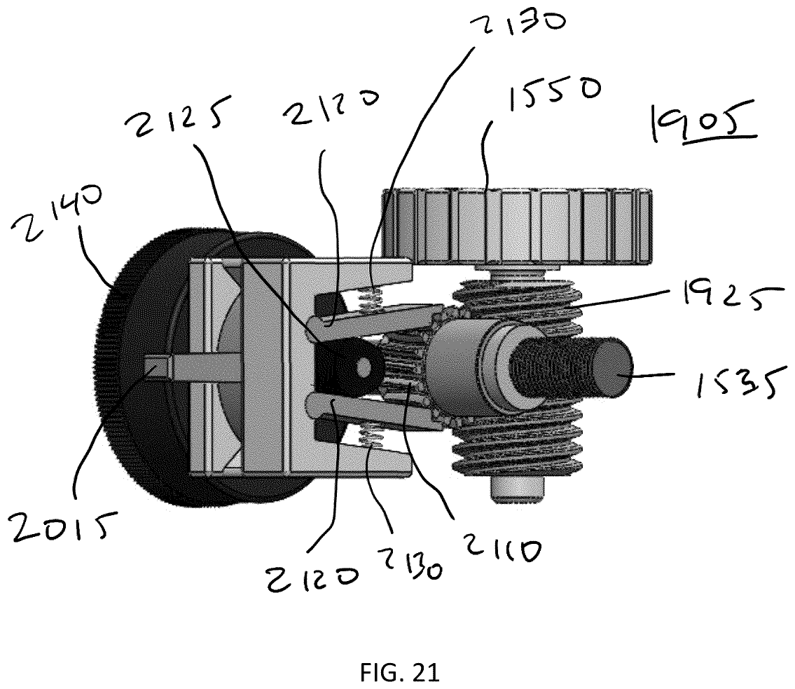

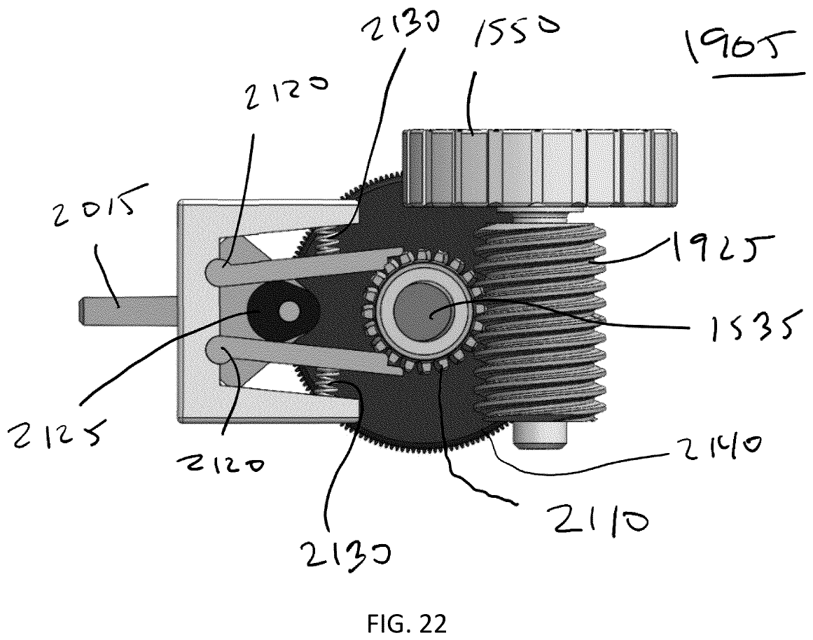

FIG. 20 shows another embodiment that includes a worm gear assembly 1905. The outer housing is not shown in FIG. 20 for clarity of illustration. FIGS. 21 and 22 show additional views of the worm gear assembly 1905. The worm gear assembly 1905 is operated via interaction with the knob 1550 and a user interface such as a toggle switch 2105. The user interface can vary in configuration and does not have to be a toggle switch. With reference to the enlarged views of FIGS. 21 and 22, a worm gear 2110 is coupled to the lead screw 1535 and to the spiral thread 1925 of the knob 1550 to achieve rotation of the lead screw 1535 via knob rotation or via actuation of the toggle switch 2105. As described below, a dial 2140 can also be used to rotate the lead screw 1535. The assembly further includes a first and second cams 2120 that are mechanically coupled, such as by being mated, to teeth of the worm gear 2110. The first and second cams include elongated arms with a first end of each arm forming a cam surface and a second end of each arm mechanically coupled to (or not coupled to) the teeth of the worm gear based upon a position of the toggle switch 2105. A third cam 2125 is positioned, such as being centered, between the first and second cams 2120 and is mechanically coupled to the toggle switch 2015. A respective spring 2130 biases each of the first and second cams 2120 toward the third cam 2125.

The toggle switch 2105 can be actuated to move a position of the third cam 2125 between the first and second cams 2120. As the toggle switch is rotated up or down, a cam surface of the third cam 2125 moves the elongated arm of each of the first or second cam upward or downward relative to the teeth of the worm gear such that the arm of the first cam locks rotation of the cam gear in one direction while the arm of the second cam is disengaged from the teeth of the cam gear thereby permitting rotation in the opposite direction. The toggle switch 2105 thus mechanically operates on the cams 2120 simultaneously. Depending on the position of the third cam 2125 (which is coupled to the toggle switch 2105), one of the two opposite cams 2120 will move upward or downward and thereby depress or constrict its respective spring 2130 to cause rotation of the worm gear 2110 in one rotational direction but not in the opposite rotational direction. This thereby causes corresponding rotation of the lead screw 1535.

The toggle switch 2015 can be configured in conjunction with a geometry and scale of the third cam 2125 to enable locking functions. For example, when the toggle switch 2105 is centered between the first and second cams, both cams engage to the teeth of the worm gear 2110 to lock all spinning orientation of the worm gear. The toggle switch 2105 can be biased in any direction to lock the rotation of the worm gear in one rotational direction or another. When of the first or second cams engages the teeth of the worm gear 2110 while the other cam is released from the worm gear, the first or second cam locks the worm gear's ability to spin in one orientation but not the other. There are thus at least three active states: In a first state, when the toggle switch is centered between the first and second cams, it locks all spinning of the worm gear. When in the second state, the arm of the first cam engages the worm gear and the arm of the second cam is disengaged from the worm gear to lock the spinning of the worm gear in one rotational direction (e.g., clockwise) while permitting or causing rotation in another direction (e.g., counterclockwise). In the third state, the arm of the second cam engages the worm gear and the arm of the first cam is disengaged from the worm gear to lock the spinning of the worm gear in one rotational direction (e.g., counterclockwise) while permitting or causing rotation in another direction (e.g., clockwise). Rotation of the worm gear in one direction can be caused by the first or second cam arm exerting a force onto the worm gear such as on a tooth of the worm gear to cause the worm gear to rotate.

The first and second cams can be configured with interfacing geometry optimized to meet the specific worm gear it is coupled to. In one implementation, the assembly includes stepped distal cam geometry that are mirrors of one another to interface with the teeth of the worm gear. The shorter end of a respective cam engages on teeth of the worm gear, while a longer end extends just over the teeth and pushes back the following teeth in the rotation to achieve the desired locking effect.

The knob 1550 can be mechanically coupled to a rail or track that extends along an axis perpendicular to a long axis of the worm gear 2110 and the lead screw 1535. The knob can slide along such a track so that it can be moved to a position where the spiral thread 1925 of the knob 1550 are properly engaged to the worm gear 2110 such as to facilitate rotations. The knob 1550 can be slidingly moved away to a position where it is not engaged with the worm gear 2110. The worm gear 2110 is thus disengaged to allow the lead screw 1535 to be spun independently and freely from the knob 1550. Such a worm gear release assembly can be manually operated by repositioning the knob 1550 from one locked position to another. Or it can be integrated into a single mechanism with the toggle switch 2015 configured to achieve a 4th active state wherein the system includes fourth cam mated to the operation of the switch itself.

The system can further include a dial 2140 that fixedly or removably attaches to the lead screw 1535. The dial 2140 can be rotated by a user to achieve 1:1 rotation of the lead screw 1535 and thereby enable quicker loading or infusing of the syringe compared to the ratios of gear reduction provided by the worm gear and the knob 1550.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A syringe adapter driver system, comprising:
a housing configured to receive a syringe device, the syringe device having a barrel and a plunger assembly including an elongated body slidably positioned inside the barrel and a seal on a distal end of the elongated body for pushing therapeutic out of the barrel via the distal coupler and into the attached therapeutic delivery device, wherein the housing has a seat that receives the barrel to secure the barrel in a fixed position on the housing;
an actuator attached to the housing, the actuator configured to drive the plunger assembly to cause the plunger assembly to extend or retract through the barrel of the syringe and expel or receive therapeutic;
a syringe driver assembly removably attaching the plunger assembly to the housing, wherein the syringe driver assembly moves relative to the housing; and
a lead screw mechanically coupling the actuator to the syringe driver assembly.

2. The syringe adapter driver system of claim 1, further comprising a gear assembly that couples the actuator to the lead screw.

3. The syringe adapter driver system of claim 2, wherein the gear assembly is a worm gear.

4. The syringe adapter driver system of claim 1, wherein the actuator is directly attached to the lead screw.

5. The syringe driver system of claim 1, wherein the actuator is a rotatable knob and wherein the predetermined volumetric flow is calibrated to an amount of rotation of the knob.

6. The syringe driver system of claim 1, wherein the rotatable knob is coupled to at least one indicator that provides an indication of predetermined volumetric flow.

7. The syringe driver system of claim 1, wherein the actuator comprises at least one gear.

8. The syringe driver system of claim 4, wherein the actuator comprises more than one gear.

9. The syringe driver system of claim 8, wherein the gears comprise a plurality of gear ratios selectable by a user.

10. The syringe driver system of claim 1, wherein the actuator comprises a lead screw.

11. The syringe driver system of claim 1, wherein the syringe driver assembly removably attaches to a plunger flange.

12. The syringe driver system of claim 1, further comprising the syringe.

13. The syringe driver system of claim 1, wherein the actuator includes a plurality of knobs and wherein each knob is configured to be rotated to achieve a specific displacement of the plunger.

14. The syringe driver system of claim 1, wherein the actuator is coupled to an automatic driver.

15. The syringe driver system of claim 11, wherein the automatic driver is a motor or a spring.

16. The syringe driver system of claim 1, wherein the actuation is calibrated to achieve a predetermined volumetric flow of the therapeutic based on an amount of actuation.

17. The syringe driver system of claim 1, wherein the actuator is coupled to a rack and pinion.

18. The syringe driver system of claim 3, further comprising a first cam having a first arm that engages the worm gear, a second cam having a second arm that engages the worm gear, and a third cam positioned between the first cam and the second cam, the third cam attached to a toggle switch.

19. The syringe driver system of claim 18, wherein movement of the toggle switch disengages the first arm or the second arm from the worm gear.
